(19) United States
(12) Patent Application Publication
(10) Pub. No.: US 2023/0187075 A1
(43) Pub. Date: Jun. 15, 2023

(54) METHOD AND SYSTEM FOR ARTIFICIAL INTELLIGENCE BASED RISK STRATIFICATION FOR GLIOMA

(71) Applicants: Shuhua Zheng, Saratoga, CA (US); Yilin Wu, Saratoga, CA (US)

(72) Inventors: Shuhua Zheng, Saratoga, CA (US); Yilin Wu, Saratoga, CA (US)

(21) Appl. No.: 17/550,312

(22) Filed: Dec. 14, 2021

(51) Int. Cl. G16H 50/30; G16H 10/60; G16H 70/60; G16H 50/70; G16H 50/20; G16B 20/20; G16B 20/10; G16B 40/20

(52) U.S. Cl. CPC G16H 50/30; G16H 10/60; G16H 70/60; G16H 50/70; G16H 50/20; G16B 20/20; G16B 20/10; G16B 40/20; G16H 50/50

(57) ABSTRACT

A method and system for machine learning based risk stratification for glioma are disclosed. The method may include obtaining clinicopathological data of a patient with a glioma and extracting biomarker data from chromosome information of the glioma of the patient. The method may further include predicting a risk stratification of the glioma based on the biomarker data and the clinicopathological data by executing a risk prediction engine. The method may further include generating a healthcare treatment recommendation for the patient based on the risk stratification of the glioma.

### TECHNICAL FIELD

This disclosure relates to artificial intelligence applications, in particular, in performing risk stratification on glioma.

### BACKGROUND

Glioma is a common type of tumor originating in the brain. High-risk low-grade glioma (LGG) should receive immediate adjuvant radiotherapy after surgical resection, whereas watchful waiting is recommended for low-risk LGG patients. However, the genetic and pathological heterogeneity of LGG complicates patient stratification for optimal treatment planning making.

### SUMMARY

This disclosure relates to systems and methods for performing risk stratification on glioma based on an artificial intelligence model.

In one aspect, provided herein is a method for risk stratification for glioma performed by a processor circuitry. The method may include obtaining clinicopathological data of a patient with a glioma and extracting biomarker data from chromosome information of the glioma of the patient. The method may further include predicting a risk stratification of the glioma based on the biomarker data and the clinicopathological data by executing a risk prediction engine and generating a healthcare treatment recommendation for the patient based on the risk stratification of the glioma.

In some embodiments, the biomarker data may include gene mutation data, chromosome variation data, or gene expression data.

In some embodiments, the biomarker data may include gene mutation data and the extracting the biomarker data from the chromosome information of the glioma of the patient may include: identifying a predetermined number of target gene types with most genetic mutations in gliomas of a plurality of patients and extracting the gene mutation data of the target gene types from the chromosome information of the glioma of the patient.

In some embodiments, the biomarker data may include chromosome variation data and the extracting the biomarker data from the chromosome information of the glioma of the patient may include: identifying a predetermined number of target gene types with most variations in a number of genes in gliomas of a plurality of patients; and extracting the chromosome variation data of the target gene types from the chromosome information of the glioma of the patient.

In some embodiments, the gene mutation data may include mutation status and mutation type of isocitrate dehydrogenase 1 (IDH1), tumor protein p53 (TP53), ATRX Chromatin Remodeler (ATRX), or capicua transcriptional repressor (CIC) and the mutation type comprises frameshift mutation, splice site mutation, missense mutation, inframe mutation, or synonymous mutation.

In some embodiments, the chromosome variation data may include copy number variations of phosphatase and tensin homolog (PTEN), Cullin 2 (CUL2), epidermal growth factor receptor (EGFR), or cyclin dependent kinase inhibitor 2A (CDKN2A).

In some embodiments, at least a portion of the chromosome variation data has a positive correlation with glioma progression-free interval and at least a portion of the chromosome variation data has a negative correlation with the glioma progression-free interval.

In some embodiments, the gene expression data may include ribonucleic acid (RNA) levels of phosphatase and tensin homolog (PTEN), Cullin 2 (CUL2), epidermal growth factor receptor (EGFR), and cyclin dependent kinase inhibitor 2A (CDKN2A).

In some embodiments, the clinicopathological data may include age of the patient at glioma diagnosis, gender of the patient, or a histological type of the patient, the histological type comprises astrocytoma, oligoastrocytoma, or oligodendroglioma.

In some embodiments, the risk prediction engine includes an artificial neural network model trained to predict risk stratification of a glioma of a patient.

In some embodiments, the method may further include obtaining the risk prediction engine by: obtaining case data of glioma cases of a plurality of patients. The case data may include clinicopathological data and biomarker data. The method may further include preprocessing the case data to obtain preprocessed case data, and training the artificial neural network model with the preprocessed case data as training data set.

In some embodiments, the preprocessing the case data may include: excluding case data of glioma cases whose longest progression-free interval or overall survival exceeds a predetermined duration threshold, converting categorical variables in the case data to indicator variables, and normalizing the case data to obtain the preprocessed case data.

In some embodiments, the method may further include: in response to a progression of the glioma of the patient based on an imaging of the glioma, determining the progression as a true progression or a pseudo progression based on the risk stratification of the glioma.

In another aspect, provided herein is a system performing risk stratification on glioma. The system may include a memory having stored thereon executable instructions and a processor circuitry in communication with the memory. When executing the instructions, the processor circuitry may be configured to obtain clinicopathological data of a patient with a glioma and extract biomarker data from chromosome information of the glioma of the patient. The processor circuitry may be further configured to predict a risk stratification of the glioma based on the biomarker data and the clinicopathological data by executing a risk prediction engine and generate a healthcare treatment recommendation for the patient based on the risk stratification of the glioma.

In some embodiments, the biomarker data may include gene mutation data, chromosome variation data, or gene expression data.

In some embodiments, the biomarker data may include gene mutation data and the processor circuitry may be further configured to: identify a predetermined number of target gene types with most genetic mutations in gliomas of a plurality of patients, and extract the gene mutation data of the target gene types from the chromosome information of the glioma of the patient.

In some embodiments, the biomarker data may include chromosome variation data and the processor circuitry may be further configured to: identify a predetermined number of target gene types with most variations in a number of chromosomes in gliomas of a plurality of patients, and extract the chromosome variation data of the target gene types from the chromosome information of the glioma of the patient.

In some embodiments, where at least a portion of the chromosome variation data has a positive correlation with glioma progression-free interval and at least a portion of the chromosome variation data has a negative correlation with the glioma progression-free interval.

In some embodiments, where the processor circuitry may be further configured to, in response to a progression of the glioma of the patient based on an imaging of the glioma, determine the progression as a true progression or a pseudo-progression based on the risk stratification of the glioma.

In another aspect, provided herein is a product performing risk stratification on glioma. The product may include a non-transitory machine-readable media and instructions stored on the machine-readable media. When being executed, the instructions may be configured to cause a processor circuitry to obtain clinicopathological data of a patient with a glioma and extract biomarker data from chromosome information of the glioma of the patient. The instructions may be further configured to cause the processor circuitry to predict a risk stratification of the glioma based on the biomarker data and the clinicopathological data by executing a risk prediction engine and generate a healthcare treatment recommendation for the patient based on the risk stratification of the glioma.

One interesting feature of the systems and methods described below may be that it may accurately identify glioma patients with high risk of progression. For example, the systems and methods may effectively identify the factors that are most relevant to the glioma progression. The factors may include clinicopathological data such as the age of the patient at glioma diagnosis, gender of the patient, and a histological type of the patient and the biomarker data such as gene mutation data of the gene types with the most mutation and chromosome variation data of the gene types with the most alterations in gene copy number. Then, the systems and methods may make use of a machine learning model to predict the risk stratification of the glioma of the patient with the factors as the input of the machine learning model. In addition, the systems and methods may further improve the accuracy of the risk stratification prediction by taking into accounts both the factors positively correlating with glioma progression and the factors negatively correlating with glioma progression in predicting the risk stratification of the glioma.

The above embodiments and other aspects and alternatives of their implementations are explained in greater detail in the drawings, the descriptions, and the claims.

### BRIEF DESCRIPTION OF THE DRAWINGS

The present disclosure may be better understood with reference to the following drawings and description. The components in the figures are not necessarily to scale. Moreover, in the figures, like-referenced numerals designate corresponding parts throughout the different views.

FIG. 1 shows an exemplary multiple-layer glioma risk stratification stack.

FIG. 2 shows an exemplary glioma risk stratification logic.

FIG. 3 shows an exemplary gene mutation chart across different age groups.

FIGS. 4A-4B show an exemplary gene copy number variations across different age groups.

FIG. 5 shows an exemplary artificial neural network model according to an embodiment of the present disclosure.

FIG. 6 shows an exemplary specific execution environment for the glioma risk stratification stack.

FIG. 7 shows a chart for prediction accuracy of the exemplary artificial neural network model in FIG. 5.

FIG. 8 shows a chart for receiver operating characteristic curve (ROC) of the exemplary artificial neural network model in FIG. 5.

FIG. 9 shows a chart for loss function of the exemplary artificial neural network model in FIG. 5.

### DETAILED DESCRIPTION

Based on the clinical trial results of Radiation Therapy Oncology Group (RTOG) 9802 and the European Organization for Research and Treatment of Cancer (EORTC) 22033-26033, adjuvant radiotherapy (RT) either alone or in combination with chemotherapy is recommended for high-risk low-grade glioma (LGG) patient. While the radiation dosages and time of intervention are relatively well established based on the EORTC 22845 and 22844 studies, the identification of high-risk LGG patients who may benefit from adjuvant RT is far from clear. In clinical practice, high-risk patients are often defined as patients with age >40 years or a less than total gross resection, the criterion adopted from the RTOG 9802 trial. As a result, only a portion of high-risk LGG patients could be identified for adjuvant RT. In addition, with emerging molecular biomarkers for LGG prognosis, LGG risk classification and corresponding treatment planning need to incorporate tumor's genetic background. Considering the genetic heterogeneity and clinicopathological variations of LGG, traditional treatment planning guidelines may fail to accurately identify high-risk LGG patients. One of the objectives of the present disclosure is to perform accurate risk stratification for glioma patients to identify patients who will benefit from immediate adjuvant RT treatment.

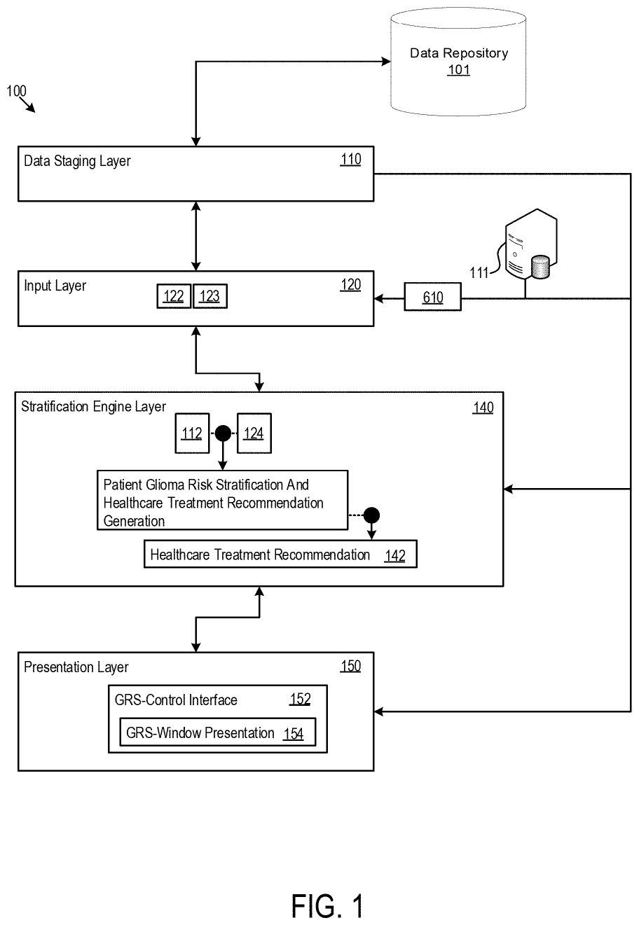

FIG. 1 shows an example multiple layer glioma risk stratification (GRS) stack 100. In this example, the GRS stack 100 includes a data staging layer 110, an input layer 120, a stratification engine layer 140, and a presentation layer 150. The GRS stack 100 may include a multiple-layer computing structure of hardware and software that may provide prescriptive analytical glioma risk stratification through data analysis.

A stack may refer to a multi-layered computer architecture that defines the interaction of software and hardware resources at the multiple layers. The Open Systems Interconnection (OSI) model is an example of a stack-type architecture. The layers of a stack may pass data and hardware resources among themselves to facilitate data processing. As one example, for the GRS stack 100, the data staging layer 110 may provide the input layer 120 with storage resources to store ingested data within a database or other data structure. In some implementations, the data staging layer 110 may be deployed as a cloud-based database platform with the capability to process mass data. Hence, the data staging layer 110 may provide a hardware resource, e.g., memory storage resources, to the input layer 120. Accordingly, the multiple-layer stack architecture of the GRS stack 100 may improve the functioning of the underlying hardware.

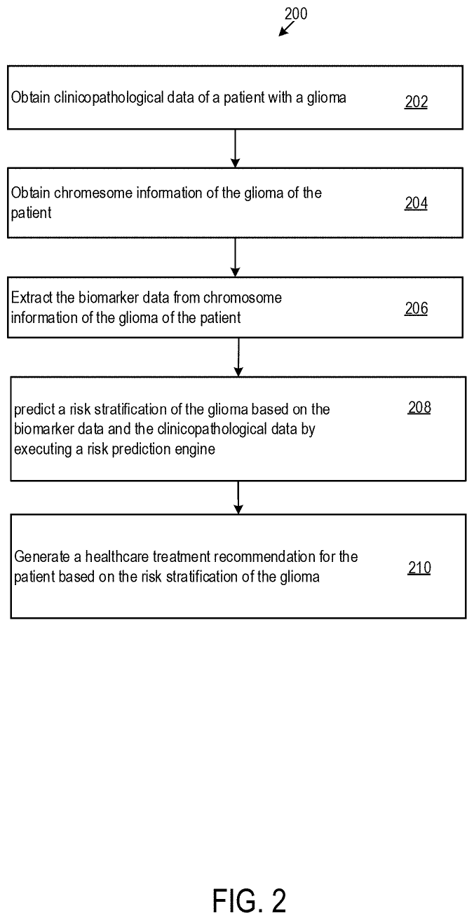

In the following, reference is made to FIG. 1 and the corresponding example GRS logic (GRL) 200 in FIG. 2. The logical features of GRL 200 may be implemented in various orders and combinations. For example, in a first implementation, one or more features may be omitted or reordered with respect to a second implementation. At the input layer 120 of the GRS stack 100, the GRL 200 may obtain clinicopathological data 122 of a patient with glioma (202). The clinicopathological data may be related to the medical information of the patient, the signs and symptoms observed by the physician, and the results of laboratory examination. For example, the clinicopathological data may include age of the patient at glioma diagnosis, gender of the patient, and a histological subtype of glioma the patient has. In the 2007 World Health Organization (WHO) classification, the main glioma subgroups classified by histological features include astrocytic tumors, oligodendroglial tumors, oligoastrocytic tumors, ependymal tumors, and neuronal and mixed neuronal-glial tumors such as gangliogliomas.

At the input layer 120 of the GRS stack 100, the GRL 200 may obtain chromosome information 123 of the glioma of the patient (204). The chromosome is a structure found in the nucleus of a cell that carries long pieces of deoxyribonucleic acid (DNA) that encodes genetic information. A chromosome contains a plurality of genes that can encode products such as ribonucleic acids (RNAs), peptides and proteins which carry out the functionalities of the chromosome information. Copy number variation (CNV) is generally defined as an amplifying or decreasing number of DNA segments that is 1 kilobase (kb) or larger in the human genome. CNV is highly associated with the development and progression of glioma, partially by impacting gene expression levels which can be measured by the messenger RNA (mRNA) levels. The chromosome information 123 of the glioma may be measured in different ways in clinical trials and clinical practices. For example, measurement of CNV may be carried by technologies such as DNA microarrays and measurement of mRNA levels may be carried by technologies such as RNA sequencing (RNA-seq).

In some cases, the clinicopathological data 122 and the chromosome information 123 may be received via communication interfaces (e.g., communication interfaces 610, discussed below). The clinicopathological data 122 and the chromosome information 123 may be accessed at least in part, e.g., via the communication interfaces 610, from data sources 111 such as a clinic database or a healthcare center data store.

At the stratification engine layer 140, the GRL 200 may utilize the clinicopathological data 122 of the patient and the chromosome information 123 of the glioma of the patient to predict the risk of the glioma and generate healthcare treatment recommendation. In an implementation, the GRL 200 may extract the biomarker data 124 from the chromosome information 123 of the glioma of the patient (206). The biomarker data may include, for example, gene mutation data, chromosome variation data, and gene expression data.

### Gene Mutation Data

The gene mutation data may include mutation status and mutation type of genes. The mutation status may indicate whether the gene is muted or not. The mutation type may include, for example, frameshift mutation, splice site mutation, missense mutation. The frameshift mutation involves either insertion or deletion of extra bases of DNA, wherein the number of bases that are either added or subtracted cannot be divisible by three. Therefore, the DNA sequence following the mutation will be disrupted or read incorrectly. The splice site mutation refers to point mutations at exon-intron boundaries and regulatory sequences recognized by RNA splicing machinery that can cause improper exon and intron recognition and may result in the formation of an abnormal mRNA transcript of the mutated gene. The missense mutation causes a single DNA base pair substitution that alters the genetic code in a way that produces an amino acid that is different from the usual amino acid at that position. The GRL 200 may identify a predetermined number of target gene types with most frequent genetic mutations in gliomas of a plurality of patients and extract the gene mutation data of the target gene types from the chromosome information of the glioma of the patient.

In an example, the GRL 200 may ingest chromosome information of gliomas of numerous patients via the communication interfaces 610 from a glioma data source such as the Cancer Genome Atlas (TCGA) datasets and store the ingested chromosome information to the data repository 101 via memory operation at the data staging layer 110. The data repository may persist data stored thereon, including for example a flat file, a relation database, and a cloud data warehouse such as Amazon Simple Storage Service (S3).

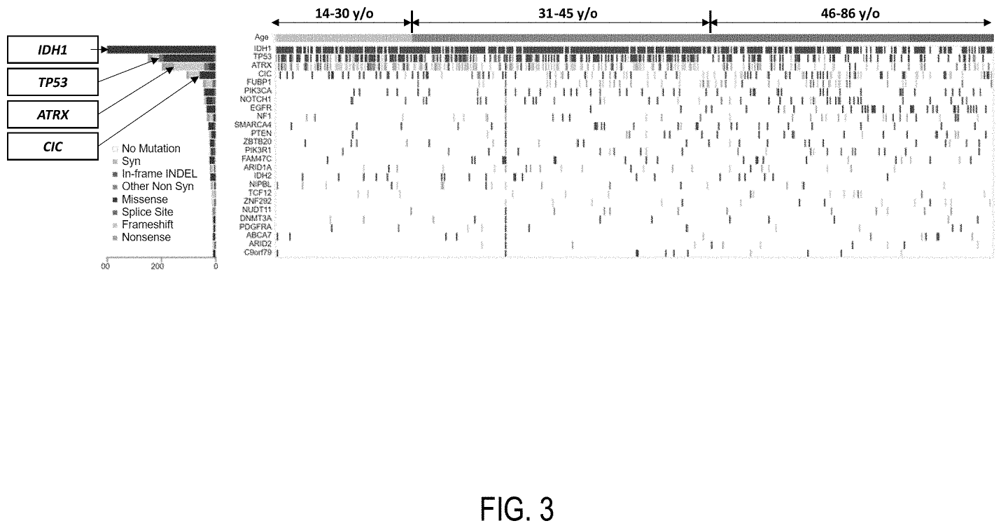

To take into account the chromosome information of gliomas for patients at different ages, the GRL 200 may create a chart of mutation of all gene types contained in the glioma chromosome per age group of patients as shown in FIG. 3 and determine the gene types with the most genetic mutations from the chart. With reference to FIG. 3, the GRL 200 may determine that the isocitrate dehydrogenase 1 (IDH1), the tumor protein p53 (TP53), the ATRX Chromatin Remodeler (ATRX), and the capicua transcriptional repressor (CIC) are the top four gene types with the most frequent gene mutations in the glioma patients.

Then, the GRL 200 may determine the target gene types according to the predetermined number of the target gene types. For example, where the predetermined number of the target gene types is three, the GRL 200 may identify the IDH1, the TP53, the ATRX and the CIC as the target gene types. Accordingly, the GRL 200 may extract the mutation status and mutation type of the IDH1, the TP53, the ATRX and the CIC from the chromosome information of the glioma of the patient under evaluation. Additionally or alternatively, the target gene types for gene mutation data extraction may be predefined and the GRL 200 may directly extract the gene mutation data of the predefined target gene types from the chromosome information of the glioma of the patient.

### Chromosome Variation Data

The chromosome variation data may include, for example, copy number variations. The copy number variation (CNV) may refer to the duplication or deletion of genes of a chromosomal region. The CNV is a type of structural variation that occurs when a gene is present in variable copy numbers compared to a reference genome. These gene CNVs can influence gene expression and can be associated with specific phenotypes and diseases. The gene expression is the process by which information from a gene is used in the synthesis of a functional gene product that enables it to produce end products such as protein or non-coding ribonucleic acid (RNA), and ultimately affect a phenotype, i.e., observable trait.

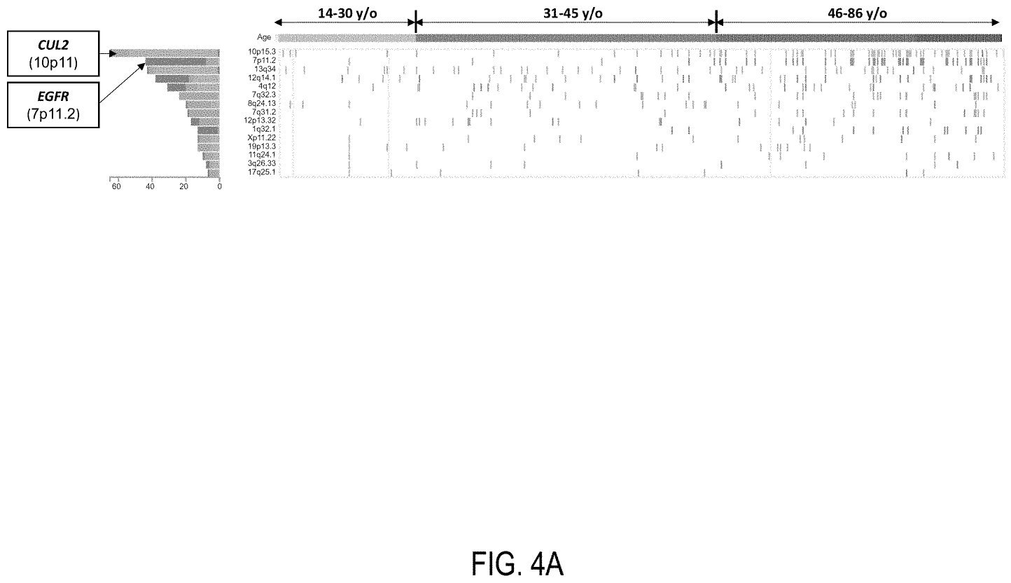

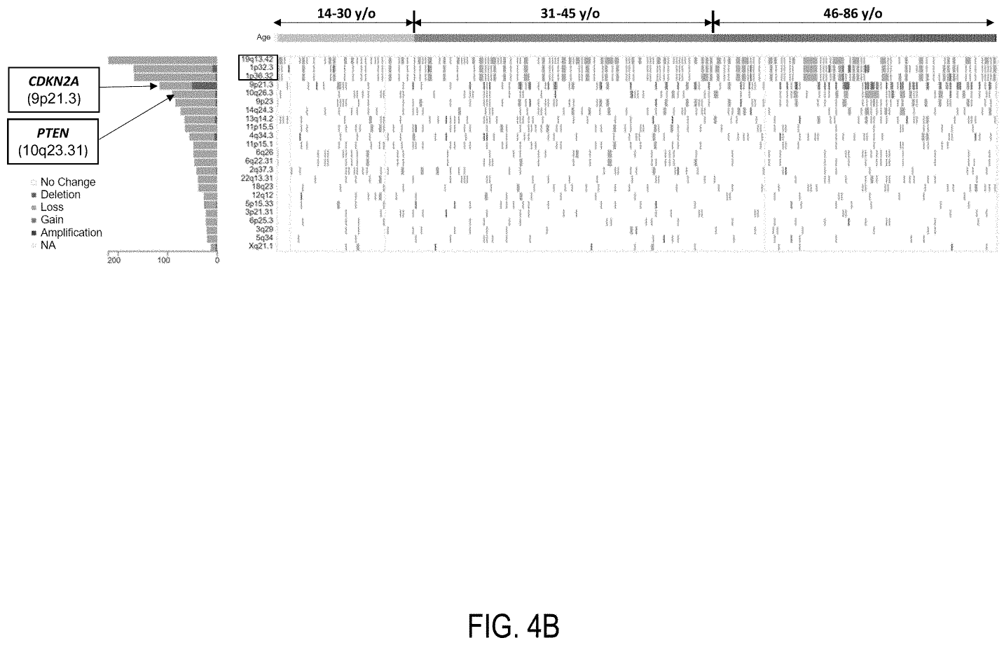

The GRL 200 may identify a predetermined number of target genes with most variations in a number of genes in gliomas of a plurality of patients and extract the chromosome variation data of the target gene types from the chromosome information of the glioma of the patient. In an example, the GRL 200 may utilize the chromosome information of gliomas of numerous patients stored in the data repository 101 to create charts of copy number variations of all gene types contained in the glioma chromosome per age group of the patients as shown in FIG. 4A and 4B. The copy number variations may include copy number gain and copy number loss. FIG. 4A illustrates genes with copy number gain while FIG. 4B illustrate genes with copy number loss. As shown, most prevalent CNVs are identified in chromosome arms 7p, 9q, 10p, 10q, 19q and 1p. Accordingly, the GRL 200 may select at least one of the gene types in the chromosome arms as the target gene types and extract the CNVs of the target gene types from the chromosome information of the glioma of the patient. Additionally or alternatively, the target gene types for CNVs extraction may be predefined and the GRL 200 may directly extract the CNVs of the predefined target gene types from the chromosome information of the glioma of the patient.

Additionally or alternatively, given that the chromosome arms 1p and 19q codel is the genetic signature of gliomas, e.g., oligodendrogliomas, the GRL 200 may filter out the genes on those bands to avoid input overlapping. As such, the GRL 200 may select the target gene types from the epidermal growth factor receptor (EGFR) (7p11.2), the cyclin dependent kinase inhibitor 2A (CDKN2A) (9p21.3), the Cullin 2 (CUL2) (10p11.21), and the phosphatase and tensin homolog (PTEN) (10q23.31). Then, the GRL 200 may extract the CNVs of the identified target gene types for CNVs from the chromosome information of the glioma of the patient.

The inventor of the present disclosure found that CNVs of some genes such as PTEN, CDKN2A, CUL2 have a positive correlation with a glioma progression-free interval (PFI) whereas CNVs of other genes such as EGFR have a negative correlation with the glioma PFI. The PFI may represent the length of time during and after the treatment of a disease, such as glioma, that a patient lives with the disease but it does not get worse. In an implementation, to take into account both the positive correlation and the negative correlation, the GRL 200 may select at least one gene positively correlating with the glioma PFI and at least one gene negatively correlating with the glioma PFI as the target gene types for CNVs extraction. For example, the GRL 200 may identify the CUL2 and the EGFR as the target gene types and extract CNVs of the CUL2 and the EGFR from the chromosome information of the glioma of the patient under evaluation.

### Gene Expression Data

In an implementation, in lieu of utilizing chromosome variation data such as CNVs of genes in the analysis of the glioma risk stratification, the GRL 200 may use the gene expression data of the identified target gene types, as discussed above with reference to FIG. 4A and 4B, as input of the glioma risk stratification analysis. The gene expression data may include, for example, ribonucleic acid (RNA) levels of the target gene types. In an example, the gene expression data may include RNA levels of PTEN, CUL2, EGFR, and CDKN2A.

Referring to FIG. 2, the GRL 200 may predict a risk stratification of the glioma based on the biomarker data and the clinicopathological data by executing a risk prediction engine (208). In an implementation, the risk stratification may be a binary classification. In this case, the risk stratification may include a high risk and a low risk. The risk prediction engine may include a machine learning model such as artificial neural network (ANN) model trained to predict risk stratification of a glioma of a patient. Machine learning is a method of data analysis that automates analytical model building. It is an application of artificial intelligence that provides the ability to automatically learn and improve from experience without being explicitly programmed.

The artificial neural network may use different layers of mathematical processing to make sense of the information it receives. Typically, an artificial neural network may have anywhere from dozens to millions of artificial neurons called units arranged in a series of layers. The input layer may receive various forms of information from the outer world. This is the data that the network aims to process or learn about. From the input layer, the data goes through one or more hidden layers. The hidden layer's job is to transform the input into something the output layer can use. The ANN may be fully connected from one layer to another. These connections are weighted. The higher the layer number is, the greater influence one layer has on another. As the data goes through each layer, the network may learn more about the data. On the other side of the network is the output layer, and this is where the network responds to the data that it was given and processed. For the ANN to learn, it should have access to a large amount of information, called a training set. For example, to train an ANN to differentiate between high-risk gliomas and low-risk gliomas, the training set would provide tagged gliomas so the network would begin to learn. Once it has been trained with the significant amount of data, it will try to classify future glioma data based on the data set throughout the different layers.

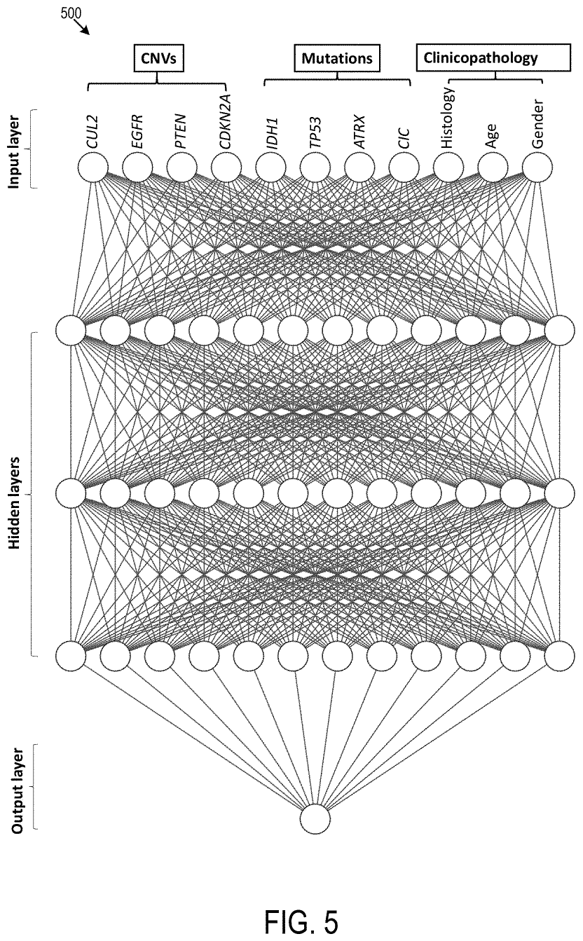

By way of example, FIG. 5 illustrates an ANN model 500 for the risk prediction engine. The ANN model 500 includes an input layer, an output layer, and two hidden layers. The GRL 200 may execute the ANN model 500 by inputting the clinicopathological data and the biomarker data such as the gene mutation data and the chromosome variation data at the input layer to obtain the predicted risk stratification of the glioma at the output layer. In this example, the clinicopathological data includes the age of the patient at glioma diagnosis, gender of the patient, and a histological type of the patient. The gene mutation data includes the mutation data of the IDH1, the TP53, the ATRX and the CIC. The chromosome variation data includes copy number variations of the CUL2, the EFGR, the PTEN, and the CDKN2A.

Subsequently, the GRL 200 may generate a healthcare treatment recommendation 142 for the patient based on the risk stratification of the glioma (210). For example, if the risk stratification of the glioma of the patient is high risk, the GRL 200 may generate the healthcare treatment recommendation of receiving immediate adjuvant radiotherapy. If the risk stratification of the glioma is low risk, the GRL 200 may generate the healthcare treatment recommendation of watchful waiting.

Additionally or alternatively, the GRL 200 may generate the healthcare treatment recommendation 142 for the patient further based on the imaging result of the glioma. Where an imaging of the glioma indicates a progression of the glioma, the GRL 200 may determine the progression as a true progression or a pseudo progression based on the predicted risk stratification of the glioma. For example, where the predicted risk stratification is high risk, the GRL 200 may determine that the progression is a true progression. Where the predicted risk stratification is low risk, the GRL 200 may determine that the progression is a pseudo progression. Then, the GRL 200 may generate the healthcare treatment recommendation of receiving adjuvant radiotherapy only in case of true progression.

Optionally, upon generating the healthcare treatment recommendation 142, the GRL 200 may execute operations at the stratification engine layer 140 to output the predicted risk stratification and the healthcare treatment recommendation for the patient in a data repository such as a cloud data warehouse. For example, the GRL 200 may store the predicted risk stratification and the healthcare treatment recommendation for the patient via a memory operation at the data staging layer 110. Additionally or alternatively, the GRL 200 may publish the predicted risk stratification and the healthcare treatment recommendation for the patient, for example, via the GRS-control interface 152 as discussed below.

Now referring to the presentation layer 150 in FIG. 1, where the GRL 200 may access the overall performance evaluation results from the stratification engine layer 140, e.g., via data staging layer 110 memory operations to generate the GRS-control interface 152 including a GRS-window presentation 154. The GRS-window presentation 154 may include, for example, data and/or selectable options related to the healthcare treatment recommendation.

The GRL 200 may train an ANN model to obtain the risk prediction engine. In an implementation, the GRL 200 may obtain case data of glioma cases of a plurality of patients. The case data may include both clinicopathological data and biomarker data of the glioma cases. The clinicopathological data may also include the PFI and the overall survival (OS) days of the glioma case. In an example, the GRL 200 may retrieve the case data of glioma cases of patients from glioma data source such as the Cancer Genome Atlas (TCGA) database. Then, the GRL 200 may preprocess the case data to obtain preprocessed case data and train the artificial neural network model with the preprocessed case data as training data.

In preprocessing the case data, the GRL 200 may exclude case data of glioma cases whose longest progression-free interval or overall survival exceeds a predetermined duration threshold. For example, the GRL 200 may exclude the case data of top 1% of the glioma cases with the longest PFI or OS days as outlier data. Additionally or alternatively, the GRL 200 may convert categorical variables in the case data to indicator variables. The categorical variables may contain label values instead of numeric values. The indicator variables may refer to numeric variables representing categorical variables. The GRL 200 may convert categorical variables to indicator variables by means of ordinal encoding, one-hot encoding, or dummy variable encoding. Then, the GRL 200 may normalize the case data to obtain the preprocessed case data. In an example, the GRL 200 may randomly assign 70% of the preprocessed case data as training data set while the remaining 30% of the preprocessed case data as validation data set. The validation data may be used to validate the prediction accuracy of the trained ANN model.

In an example , the ANN model is constructed with the Keras Python library, which is an open-source software library that provides a Python interface for artificial neural networks. The argument “Dense” is deployed for each layer with activation function “relu” for all the hidden layers. The functions “sigmoid” and “adam” are chosen as the activation function and optimizer, respectively, for the output layer. The loss function is fetched with the “binary_crossentropy” command. The “early_stop” and accuracy functions are deployed to prevent overfitting and evaluate the model's performance, respectively. Accuracy and loss function for both the training data set and the validation data set are plotted for each epoch.

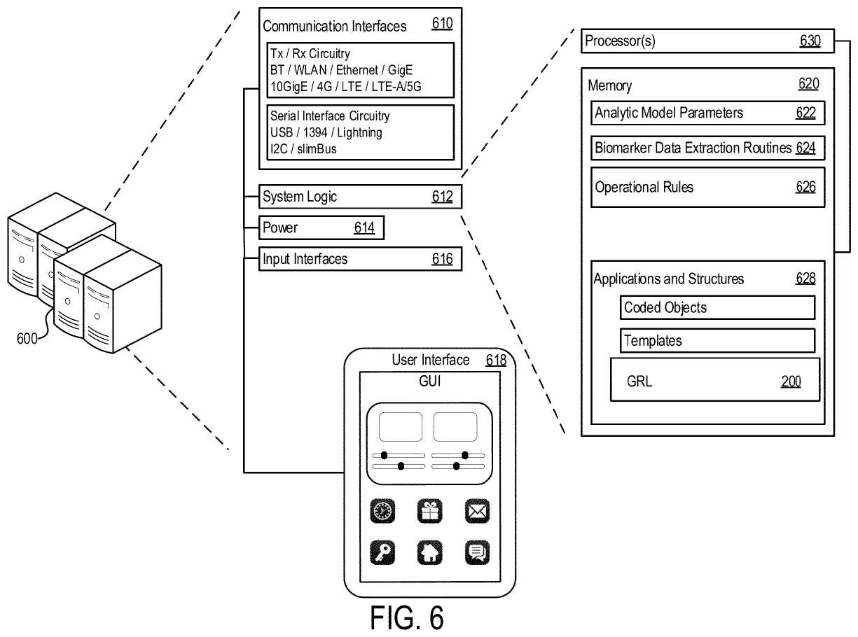

FIG. 6 shows an example specific execution environment 600 for the GRS stack 100 described above. The execution environment 600 may include system logic 612 to support execution of the multiple layers of GRS stack 100 described above. The system logic may include processors 630, memory 620, and/or other circuitry.

The memory 620 may include analytic model parameters 622, biomarker data extraction routines 624, and operational rules 626. The memory 620 may further include applications and structures 628, for example, coded objects, machine instructions, templates, or other structures to support extracting biomarker data, predicting glioma risk stratification, generating healthcare treatment recommendation, or other tasks described above. The applications and structures may implement the GRL 200.

The execution environment 600 may also include communication interfaces 610, which may support wireless, e.g. Bluetooth, Wi-Fi, WLAN, cellular (4G, LTE/A, 5G), and/or wired, Ethernet, Gigabit Ethernet, optical networking protocols. The communication interfaces 610 may also include serial interfaces, such as universal serial bus (USB), serial ATA, IEEE 1394, lighting port, I²C, slimBus, or other serial interfaces. The communication interfaces 610 may be used to support and/or implement remote operation of the GRS-control interface 152. The execution environment 600 may include power functions 614 and various input interfaces 616. The execution environment may also include a user interface 618 that may include human-to-machine interface devices and/or graphical user interfaces (GUI). The user interface 618 may be used to support and/or implement local operation of the GRS-control interface 152. In various implementations, the system logic 612 may be distributed over one or more physical servers, be implemented as one or more virtual machines, be implemented in container environments such as Cloud Foundry or Docker, and/or be implemented in Serverless (functions as-a-Service) environments.

In some cases, the execution environment 600 may be a specially defined computational system deployed in a cloud platform. In some cases, the parameters defining the execution environment may be specified in a manifest for cloud deployment. The manifest may be used by an operator to requisition cloud-based hardware resources, and then deploy the software components, for example, the GRS stack 100, of the execution environment onto the hardware resources. In some cases, a manifest may be stored as a preference file such as a YAML (yet another mark-up language), JSON, or other preference file type.

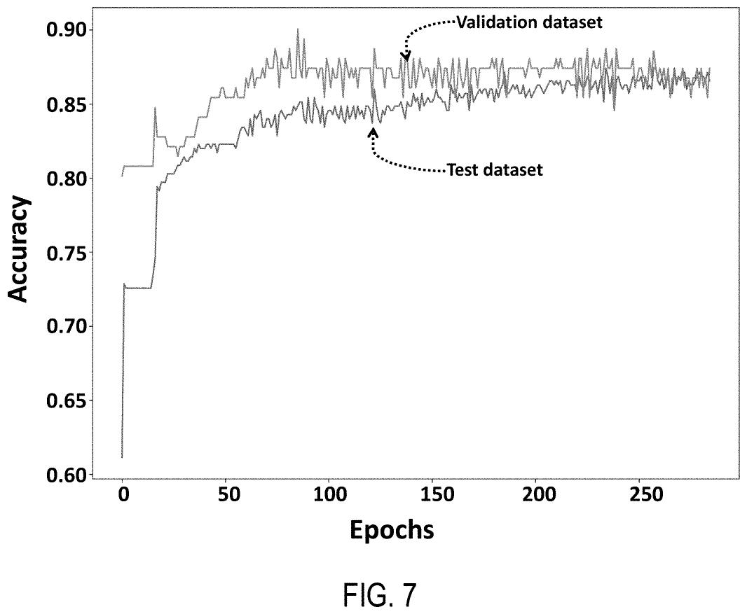

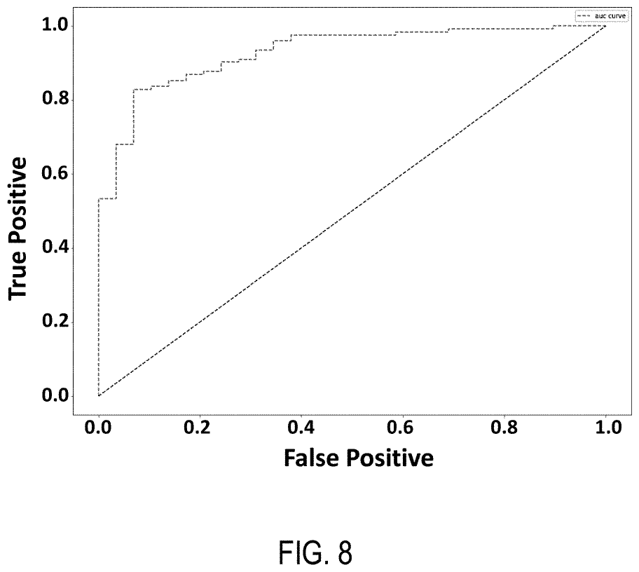

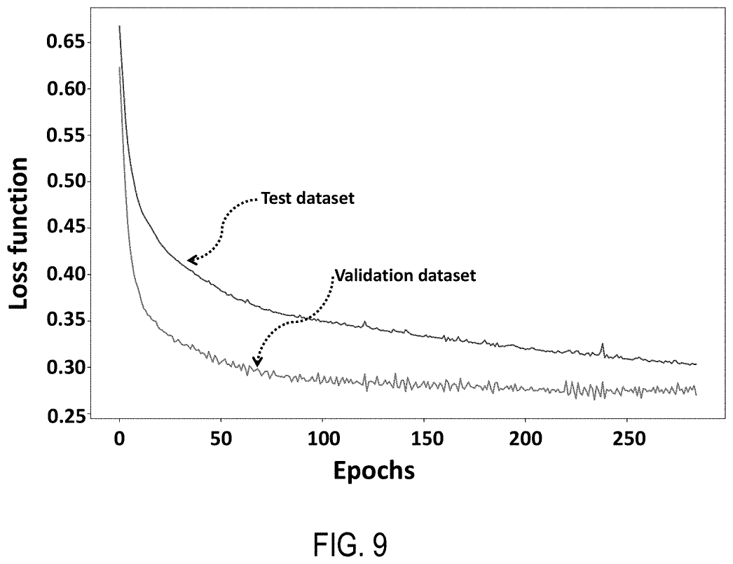

FIGS. 7-9 illustrate performance test results of the ANN model 500 based on the training data set and the validation data set. The chart in FIG. 7 shows that, with the increase of the training epochs, the prediction accuracy of the ANN model 500 can reach 90%. The chart in FIG. 8 shows that the area under the ROC curve (AUC) score can reach 0.9. In addition, as shown in FIG. 9, the loss function of the validation set reaches a minimal value after about 200 epochs, which indicates the model has been sufficiently trained and overfitting was prevented. Here, an epoch refers to one cycle through the full training dataset by the ANN model.

The methods, devices, processing, circuitry, and logic described above may be implemented in many different ways and in many different combinations of hardware and software. For example, all or parts of the implementations may be circuitry that includes an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof. The circuitry may include discrete interconnected hardware components or may be combined on a single integrated circuit die, distributed among multiple integrated circuit dies, or implemented in a Multiple Chip Module (MCM) of multiple integrated circuit dies in a common package, as examples.

Accordingly, the circuitry may store or access instructions for execution, or may implement its functionality in hardware alone. The instructions may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CD-ROM), Hard Disk Drive (HDD), or other magnetic or optical disk; or in or on another machine-readable medium. A product, such as a computer program product, may include a storage medium and instructions stored in or on the medium, and the instructions when executed by the circuitry in a device may cause the device to implement any of the processing described above or illustrated in the drawings.

The implementations may be distributed. For instance, the circuitry may include multiple distinct system components, such as multiple processors and memories, and may span multiple distributed processing systems. Parameters, databases, and other data structures may be separately stored and managed, may be incorporated into a single memory or database, may be logically and physically organized in many different ways, and may be implemented in many different ways. Example implementations include linked lists, program variables, hash tables, arrays, records (e.g., database records), objects, and implicit storage mechanisms. Instructions may form parts (e.g., subroutines or other code sections) of a single program, may form multiple separate programs, may be distributed across multiple memories and processors, and may be implemented in many different ways. Example implementations include stand-alone programs, and as part of a library, such as a shared library like a Dynamic Link Library (DLL). The library, for example, may contain shared data and one or more shared programs that include instructions that perform any of the processing described above or illustrated in the drawings, when executed by the circuitry.

What is claimed is:

1. A method comprising:
obtaining, with a processor circuitry, clinicopathological data of a patient with a glioma;
extracting, with the processor circuitry, biomarker data from chromosome information of the glioma of the patient;
predicting, with the processor circuitry, a risk stratification of the glioma based on the biomarker data and the clinicopathological data by executing a risk prediction engine; and
generating, with the processor circuitry, a healthcare treatment recommendation for the patient based on the risk stratification of the glioma.

2. The method of claim 1, where the biomarker data comprises gene mutation data, chromosome variation data, or gene expression data.

3. The method of claim 2, where the biomarker data comprises gene mutation data and the extracting the biomarker data from the chromosome information of the glioma of the patient comprises:
identifying a predetermined number of target gene types with most genetic mutations in gliomas of a plurality of patients; and
extracting the gene mutation data of the target gene types from the chromosome information of the glioma of the patient.

4. The method of claim 2, where the biomarker data comprises chromosome variation data and the extracting the biomarker data from the chromosome information of the glioma of the patient comprises:
identifying a predetermined number of target gene types with most variations in a number of genes in gliomas of a plurality of patients; and
extracting the chromosome variation data of the target gene types from the chromosome information of the glioma of the patient.

5. The method of claim 2, where the gene mutation data comprises mutation status and mutation type of isocitrate dehydrogenase 1 (IDH1), tumor protein p53 (TP53), ATRX Chromatin Remodeler (ATRX), or capicua transcriptional repressor (CIC) and the mutation type comprises frameshift mutation, splice site mutation, missense mutation, inframe mutation, or synonymous mutation.

6. The method of claim 2, where the chromosome variation data comprises copy number variations of phosphatase and tensin homolog (PTEN), Cullin 2 (CUL2), epidermal growth factor receptor (EGFR), or cyclin dependent kinase inhibitor 2A (CDKN2A).

7. The method of claim 6, where at least a portion of the chromosome variation data has a positive correlation with glioma progression-free interval and at least a portion of the chromosome variation data has a negative correlation with the glioma progression-free interval.

8. The method of claim 2, where the gene expression data comprises ribonucleic acid (RNA) levels of phosphatase and tensin homolog (PTEN), Cullin 2 (CUL2), epidermal growth factor receptor (EGFR), and cyclin dependent kinase inhibitor 2A (CDKN2A).

9. The method of claim 1, where the clinicopathological data comprises age of the patient at glioma diagnosis, gender of the patient, or a histological type of the patient, the histological type comprises astrocytoma, oligoastrocytoma, or oligodendroglioma.

10. The method of claim 1, where the risk prediction engine includes an artificial neural network model trained to predict risk stratification of a glioma of a patient.

11. The method of claim 10, where the method further comprises obtaining the risk prediction engine by:
obtaining case data of glioma cases of a plurality of patients, the case data comprises clinicopathological data and biomarker data;
preprocessing the case data to obtain preprocessed case data; and
training the artificial neural network model with the preprocessed case data as training data set.

12. The method of claim 11, where the preprocessing the case data comprises:
excluding case data of glioma cases whose longest progression-free interval or overall survival exceeds a predetermined duration threshold;
converting categorical variables in the case data to indicator variables; and
normalizing the case data to obtain the preprocessed case data.

13. The method of claim 1, where the method further comprises:
in response to a progression of the glioma of the patient based on an imaging of the glioma, determining the progression as a true progression or a pseudo progression based on the risk stratification of the glioma.

14. A system, comprising:
a memory having stored thereon executable instructions;
a processor circuitry in communication with the memory, the processor circuitry when executing the instructions configured to:
obtain clinicopathological data of a patient with a glioma;
extract biomarker data from chromosome information of the glioma of the patient;
predict a risk stratification of the glioma based on the biomarker data and the clinicopathological data by executing a risk prediction engine; and
generate a healthcare treatment recommendation for the patient based on the risk stratification of the glioma.

15. The system of claim 14, where the biomarker data comprises gene mutation data, chromosome variation data, or gene expression data.

16. The system of claim 15, where the biomarker data comprises gene mutation data and the processor circuitry is configured to:
identify a predetermined number of target gene types with most genetic mutations in gliomas of a plurality of patients; and
extract the gene mutation data of the target gene types from the chromosome information of the glioma of the patient.

17. The system of claim 15, where the biomarker data comprises chromosome variation data and the processor circuitry is configured to:
identify a predetermined number of target gene types with most variations in a number of chromosomes in gliomas of a plurality of patients; and
extract the chromosome variation data of the target gene types from the chromosome information of the glioma of the patient.

18. The system of claim 15, where at least a portion of the chromosome variation data has a positive correlation with glioma progression-free interval and at least a portion of the chromosome variation data has a negative correlation with the glioma progression-free interval.

19. The system of claim 14, where the processor circuitry is further configured to:
in response to a progression of the glioma of the patient based on an imaging of the glioma, determine the progression as a true progression or a pseudo-progression based on the risk stratification of the glioma.

20. A product, comprising:
a non-transitory machine-readable media; and
instructions stored on the machine-readable media, the instructions configured to, when executed, cause a processor circuitry to:
obtain clinicopathological data of a patient with a glioma;
extract biomarker data from chromosome information of the glioma of the patient;
predict a risk stratification of the glioma based on the biomarker data and the clinicopathological data by executing a risk prediction engine; and
generate a healthcare treatment recommendation for the patient based on the risk stratification of the glioma.
